(19) United States
(12) Patent Application Publication
(10) Pub. No.: US 2011/0236461 A1
(43) Pub. Date: Sep. 29, 2011

(54) METHODS FOR STIMULATING CHONDROGENESIS UTILIZING A POTASSIUM CHANNEL INHIBITOR

(76) Inventor: T. Michael Underhill, Vancouver (CA)

(21) Appl. No.: 13/058,505

(22) PCT Filed: Aug. 12, 2009
(86) PCT No.: PCT/CA09/01125

§ 371 Date: Jun. 9, 2011

Related U.S. Application Data
(60) Provisional application No. 61/088,400, filed on Aug. 13, 2008.

(51) Int. Cl. A61K 35/32; A61K 31/245; A61K 9/00; A61K 31/4409; A61K 31/4015; A61K 31/198; A61K 39/395; A61K 31/202; A61K 38/18; A61K 38/17; A61K 31/728; A61K 31/7008; A61K 31/737; A61P 19/04; A61P 19/02; A61P 19/00

(52) U.S. Cl. 424/426; 514/535; 424/93.7; 424/423; 514/352; 514/423; 514/563; 424/130.1; 514/560; 424/400; 514/537; 514/8.8; 514/7.6; 514/9.1; 514/17.1; 514/54; 514/62

(57) ABSTRACT

The invention is directed to a method for stimulating chondrogenesis comprising administering to a subject in need thereof a composition comprising a therapeutically effective amount of a potassium channel inhibitor.

This application claims the benefit of priority to U.S. Provisional Patent Application No. 61/088,400, filed Aug. 13, 2008, the entirety of which is incorporated by reference.

The multistep process of chondrogenesis or cartilage formation plays a critical role in skeletal development and maintenance. Chondrogenesis begins when mesenchymal cells committed to the chondrogenic lineage aggregate together to form cell clusters, or condensations. In the center of the condensations, prechondrocytes emerge and turn off expression of mesenchymal and condensation markers, and increase the expression of Col2a1 and other early cartilage markers. Differentiation of these prechondrocytes is accompanied by increased expression of Col2a1 and other extracellular matrix proteins, including but not limited to Aggrecan and other collagens. Under certain conditions, the chondrocytes within cartilage undergo maturation into hypertrophic chondrocytes. Once fully differentiated, hypertrophic cells become surrounded by a calcified extracellular matrix and die through apoptosis as the cartilage matrix is replaced by bone. Karsenty and Wagner, Developmental Cell, 2:389-406 (2002).

Nonhypertrophic chondrocytes include reserve and proliferating chondrocytes. They are considered to be the cartilage-forming cells because they express α1(II) collagen and aggrecan, the major components of cartilaginous extracellular matrix. The transcription factor Sox9 is essential for differentiation of mesenchymal cells into chondrocytes at the mesenchymal condensation stage. Sox5 and Sox6 are co-expressed with Sox9 in nonhypertrophic chondrocytes. Increased expression and/or activity of Sox5, Sox6, and/or Sox9 is associated with the maintenance of a chondrocytic phenotype, whereas these genes are down-regulated during chondrocyte hypertrophy. Thus, increased Sox5, Sox6, and/or Sox9 expression and/or activity is thought to be associated with inhibition of chondrocyte hypertrophy.

Healthy cartilage is crucial for joint function, as it protects bones from load-bearing forces, and allows for joint motion. In addition, the precursor cells responsible for cartilage formation also participate in bone formation. Articular or hyaline cartilage is a highly specialized tissue, consisting of chondrocytes embedded in a network of extracellular matrix components such as collagens and proteoglycans. The chondrocytes maintain cartilage architecture by performing both formation and breakdown of critical extracellular matrix components. Steinert et al., Arthritis Research & Therapy 9(3):213 (2007).

Numerous factors and pathways have been shown to play an important role in regulating the process of chondrogenesis, and disruptions in the process can lead to severe developmental consequences. In adults, impairment of chondrocyte function can cause cartilage degradation and osteoarthritis. Also, because cartilage exists as an avascular tissue, articular cartilage has a limited spontaneous repair response when damaged by trauma or disease processes. In many cases, progenitor cells that could facilitate tissue repair do not migrate to the damaged site, and defects can remain permanently.

Currently, the most common methods for cartilage repair involve surgical procedures such as autologous chondrocyte transplantation and delivery of matrices seeded with chondrogenic cells or chondrogenic factors, among other procedures. While several of these surgical methods have met with short-term success, long-term clinical results have not yet been achieved, and a critical need still exists for pharmacological agents that promote articular cartilage formation, healing and maintenance. Known mediators of chondrogenesis include members of the transforming growth factor β (TGF-β) superfamily such as bone morphogenic proteins (BMPs) and fibroblast growth factors (FGFs). In addition, transcription factors have been considered for use in promoting chondrogenesis. However, these may not be ideal for therapeutic applications due to stability or delivery limitations.

The invention is based in part on the surprising discovery that compounds that bind to and/or inhibit potassium channels can be administered to stimulate chondrogenesis in a subject. Accordingly, compositions comprising specific or general potassium channel inhibitors are described for use in methods to promote chondrogenesis. In some embodiments, the potassium channel inhibitor inhibits the Kv classes of potassium channel, such as, e.g., butamben. In some embodiments, the potassium channel inhibitor is a broad spectrum potassium channel blocker, such as, e.g., 4-aminopyridine (4-AP).

Pharmaceutical compositions are disclosed comprising a pharmaceutically effective amount of a potassium channel inhibitor capable of inducing chondrogenesis and a pharmaceutically acceptable excipient. In some embodiments, the pharmaceutical composition includes a therapeutically effective amount of at least one other therapeutic agent, and a pharmaceutically acceptable excipient. In exemplary embodiments, the other therapeutic agent is a second chondrogenesis stimulating agent. In other embodiments, the other therapeutic agent is a chondrogenic protective agent. In certain embodiments, the composition comprising a potassium channel inhibitor will also include a second chondrogenesis stimulating agent and a chondrogenic protective agent.

Methods of stimulating chondrogenesis in a subject by administering a composition comprising a potassium channel inhibitor are provided. The invention further provides methods of treating a cartilage pathology, cartilage trauma, or a chondrogenic disease in a subject. Cartilage pathologies and chondrogenic diseases include, e.g., chondromalacia, chondrodysplasias, osteochondritis, degenerative joint disease, fibrotic joint disease, rheumatoid arthritis, osteoarthritis, polychondritis and artificial articulation. Certain embodiments of the invention provide methods of treating or repairing cartilage defects such as, e.g., articular cartilage tears, congenital cartilage defects, and cartilage injury caused by bone fractures.

In certain embodiments, the composition is administered systemically, such as, by intravenous injection, while in other embodiments, the composition is administered locally. Local administration methods include direct injection, topical administration, and device or implant coatings. Some embodiments of the invention include administering the composition via implantation. The composition may further involve a carrier such as a matrix or device, or may be encapsulated or injected in a viscous form for delivery to the site of tissue damage. When administered, the therapeutic composition is in a pyrogen-free, physiologically acceptable form.

Other embodiments of the invention are discussed throughout this application. Other objects, features, and advantages of the present invention will become apparent from the following detailed description. Any embodiment discussed with respect to one aspect of the invention is intended to apply to other aspects of the invention.

### BRIEF DESCRIPTION OF THE DRAWINGS

FIG. 1 shows expression of Kcnd2 and Sox9 in mouse limb bud sections using RT-qPCR. E11.5 limb buds were serially sectioned and each region was transferred to individual microfuge tubes containing 700 ml of RLT lysis buffer (Qiagen RNeasy kit). After homogenizing the sections in RLT lysis buffer by repeated pipetting, RNA was isolated according to the manufacturer's protocol. Gene expression was analyzed using RT-qPCR according to the manufacturer's instructions (Applied Biosystems Inc.).

FIG. 2 shows the results of siRNA knockdown of Kcnd2, and its effects on Sox9 expression. Kcnd2 and Sox9 expression were measured by RT-qPCR.

FIG. 3A shows treatment of primary limb mesenchymal (PLM) cultures with various doses of butamben. The x-axis indicates butamben doses, and the y-axis represents percent stimulation of pGL3(4×48) reporter gene compared to control cultures. FIG. 3B demonstrates the ability of butamben to increase Sox9 expression in PLM cultures relative to control. Sox9 expression was measured by RT-qPCR after butamben treatment for 1 or 3 days.

FIG. 4A shows treatment of PLM cultures with benzocaine analgesics. The y-axis represents percent stimulation of pGL3(4×48) reporter gene compared to control cultures. FIG. 4B displays chemical structures for butamben and the structurally related analgesic benzocaine.

FIG. 5 shows treatment of PLM cultures with the broad-spectrum potassium channel inhibitor 4-aminopyridine (4-AP). The x-axis indicates 4-AP doses, and the y-axis represents percent stimulation of pGL3(4×48) reporter gene compared to control cultures.

FIG. 6 shows the activity of the pGL3(4×48) reporter in murine limb bud-derived cells in response to different treatment combinations. The x-axis indicates the different treatments and the y-axis represents percent stimulation of the pGL3(4×48) reporter construct relative to the control culture. BAB=butamben, 4310=pan RAR antagonist 4310, B4=bone morphogenetic protein-4 (BMP-4). Reporter gene activity is the average of three replicates.

FIG. 7 shows Mmp13 mRNA levels in treated murine limb bud-derived cell cultures, as measured by a TaqMan® real-time PCR assay. Mmp13 expression levels are expressed as a percent of control cells' expression level on day 5. BAB=butamben, 4310=pan RAR antagonist 4310, B4=bone morphogenetic protein-4 (BMP-4). Expression levels are the average of two replicates.

### DEFINITIONS

The term “stimulating chondrogenesis” or “promoting chondrogenic activity” as used herein, includes increasing differentiation of mesenchymal cells into chondrocytes, maintaining the nonhypertrophic chondrocyte phenotype by inhibiting their differentiation into hypertrophic chondrocytes, increasing cartilage formation, enhancing the production of cartilage matrix, and/or inducing cartilage repair. A “chondrogenesis stimulating agent” is a compound that stimulates chondrogenesis.

A “chondrogenic protective agent” or “chondroprotective agent” is a compound that inhibits degeneration of cartilage. Examples of suitable chondroprotective agents include IL-1 receptor antagonists, TNF receptor antagonists, COX-2 inhibitors, viscosupplements (i.e. hyaluronic acid), glucosamine, or chondroitin sulphate. Additional chondrogenic protective agents are described in detail at columns 12 to 14 of U.S. Pat. No. 7,067,144, incorporated herein by reference.

A “cartilage defect” includes cartilage damage caused by injury, disease, or improper formation of cartilage during development. The cartilage defect may be caused by an injury to bone as well as cartilage, such as bone fractures or osteochondral defects. The cartilage defect may also result from surgical procedures.

The terms “treatment,” “therapeutic method,” and their cognates refer to treatment or prophylactic/preventative measures. Those in need of treatment may include individuals already having a particular medical disorder as well as those who may ultimately acquire the disorder.

The terms “therapeutically effective dose,” or “therapeutically effective amount,” refer to the amount of potassium channel inhibitor in a composition that results in prevention or delay of onset or amelioration of symptoms of cartilage damage or defects in a subject or an attainment of a desired biological outcome, such as increased chondrogenesis. The effective amount can be determined by methods well-known in the art and will vary with the nature and severity of the condition treated.

A “subject” can be a mammal, e.g., a human, primate, ovine, bovine, porcine, equine, feline, canine, and a rodent (rat or mouse).

The use of the word “a”, “an” or “the” when used in conjunction with the term “comprising” in the claims and/or the specification may mean “one,” but it is also consistent with the meaning of “one or more,” “at least one,” and “one or more than one.”

### EXEMPLARY EMBODIMENTS

### Chondrogenesis Assays

Potassium channel inhibitors useful in the methods of the invention possess the ability to stimulate chondrogenesis in a subject. This activity may be evaluated using any one of a number of assays known in the art for measuring the chondrogenic potential of compounds. These include both of in vivo and in vitro systems, such as, e.g., model systems for measuring chondrogenesis using primary limb mesenchymal cultures (Weston et al., J. Cell. Biol. 158(1):39-51 (2002)), high-density micromass clutures prepared from limb buds (Pizette et al., Dev. Biol., 219:237-249 (2000); Chimal-Monroy et al., Developmental Dynamics, 224:314-320 (2002)), and animal models (Lin et al., Arthritis & Rheumatism, 58(4):1067-75 (2008)), among others.

Sox9 and related transcription factors Sox5 and Sox6 have been identified as essential for chondrogenic differentiation and cartilage formation. Sox9 is required for both commitment and differentiation of chondroblasts. The onset of chondroblast differentiation is associated with increased expression of L-Sox5 and Sox6, and together with Sox9, they increase the expression of Col2a1 and other chondrogenic genes. Thus, many art recognized assays for evaluating chondrogenic activity involve monitoring for induction of one or more of these genes. One example of a reliable means for assessing the status of chondroblast differentiation is the use of primary limb mesenchymal (PLM) cultures transfected with a firefly luciferase based reporter gene derived from the Col2a1 gene containing binding sites for Sox5, Sox6, and Sox9. (Hoffman et al., J. Cell. Biol. 174:101-13 (2006); Lefebvre et al., Mol. Cell. Biol. 17:2336-46 (1997)).

Because an inverse relationship exists between the activity of the retinoid signaling pathway and chondrocyte differentiation, an alternate method of evaluating chondrogenic activity is to monitor the activity of the retinoid pathway with a retinoic acid responsive reporter gene. (Weston 2002).

### Chondrogenic Compositions Comprising Potassium Channel Inhibitors

Potassium channels are expressed in eukaryotic and prokaryotic cells, and modulate a number of cellular events such as muscle contraction, neuro-endocrine secretion, frequency and duration of action potentials, electrolyte homeostasis, and resting membrane potential. Potassium channels have been classified according to their biophysical and pharmacological characteristics, and subclasses have been named based on amino acid sequence and functional properties. In particular, voltage gated potassium channels (e.g. Kv1, Kv2, Kv3, Kv4 and Kcne) play important roles in many biological processes. Subtypes within these subclasses have been characterized based on function, pharmacology and distribution in cells and tissues.

Many types of potassium channel inhibitors are known in the art, and include both broad-spectrum inhibitors and compounds that specifically inhibit certain potassium channels. For example, in certain embodiments of the invention, the potassium channel inhibitor inhibits Kv1 and 4 classes (Winkelman et al. J. Pharmacol. Exp. Ther. 314:1177-86 (2005); Beekwilder et al., J. Pharmacol. Exp. Ther. 304:531-8 (2003)). The Kv4.2 potassium channel encoded by Kcnd2 is also blocked by butamben (Winkelman et al. J. Pharmacol. Exp. Ther. 314:1177-86 (2005)), and knockdown of Kcnd2 in PLM cultures significantly induces Sox9 expression.

Thus, in one embodiment of the invention, the potassium channel inhibitor acts by inhibiting the Kcnd2 expression product, such as, e.g., a neutralizing antibody to Kv4.2, butamben, arachidonic acid (Holmqvist et al., J. Neurosci., 21(12):4154-61 (2001)), peptides disclosed in U.S. Pat. No. 7,078,481 (incorporated by reference), and compounds disclosed in PCT Publication 2007/138112 and U.S. Pat. No. 7,105,534 (both incorporated by reference), among others. Although it is possible that BMPs may function in part through modulation of potassium channel activity (and particularly Kcnd2) to regulate expression of the chondroblastic phenotype, BMPs are not considered to be potassium channel inhibitors for the purposes of this invention.

Kcnd2 nucleic acid and protein sequences from a variety of species, including human, mouse, rat, macaque, chimp, chicken, and zebrafish, are known in the art. These sequences can be retrieved from, for example, the NCBI web portal using the following GeneIDs: 3751, 16508, 65180, 574361, 472489, 374143, and 570188, respectively. For example, the human, mouse, and rat GeneIDs can be used to retrieve Kcnd2 protein (NP_{—}036413.1, NP_{—}062671.1, and NP_{—}113918.2) and mRNA (NM_{—}012281.2, NM_{—}019697.3, and NM_{—}031730.2) sequences. Similar data for other classes of potassium channels are also publically available. All information associated with all GeneIDs and reference protein and nucleic acid sequences referenced in this application, including their associated annotations, is incorporated by reference.

In some embodiments, a potassium channel inhibitor is administered in an amount effective to inhibit Kv4.2 activity by at least 5, 10, 15, 20, 25, 30, 50, 60, 80, or 90%; or 2, 3, 4, 5, 10, 20, 40, or 80-fold. All ranges of parameters in this application should be understood to also describe sub ranges bounded by these values, e.g., at least 10, 20, or 40 describes 10 to 20, 10 to 40, and 20 to 40. In still other embodiments, Kcnd2, or another gene encoding a potassium channel, is inhibited at the RNA or protein level by means known in the art, e.g., small interfering RNA or neutralizing antibodies. In these embodiments, mRNA and/or protein expression levels are reduced by at least 5, 10, 15, 20, 25, 30, 50, 70, 90%; or 2, 3, 4, 5, 10, 20, 40, or 80 fold, relative to untreated controls. In some embodiments, the potassium channel is inhibited at the protein or RNA level in an amount sufficient to increase mRNA, protein or activity levels of Sox 5, Sox 6, or Sox 9 (human GeneIDs 6660, 55553, and 6662, respectively) by at least 5, 10, 15, 20, 25, 30, 50, 70, or 90%; or 2, 4, 8, 10, 20, 40, or 80-fold.

In other embodiments, the potassium channel inhibitor is 4-AP. In some embodiments, the potassium channel inhibitor is glimepiride. Another exemplary potassium channel inhibitor for use in the methods of the invention includes nateglinide. Numerous other potassium channel blockers have been identified, and many are commercially available. See, e.g., charybdotoxin derivatives described in U.S. Pat. No. 5,006,512, and small molecules inhibitors disclosed in U.S. Pat. Nos. 6,083,986, 6,303,637, 6,395,730, 6,706,720, 6,870,055, and 7,137,248, among others.

Still other potassium channel inhibitors may be discovered in the future and used in the methods of the invention. For example, European Patent 1506227, incorporated herein by reference, discloses methods of identifying inhibitors of the Kv4 potassium channel family. The skilled artisan can also use available sequences and solved biological structures, including crystal and NMR structures, for a target potassium channel, such as the Kcnd2 gene product (see, for example, mmdbIDs 26610 and 24306), for rational drug selection or design.

In some embodiments, chondrogenic compositions for use in the methods of the invention comprise a pharmaceutically effective amount of a potassium channel inhibitor capable of inducing chondrogenesis and a pharmaceutically acceptable excipient. Suitable excipients are well known in the art. In some embodiments, the pharmaceutical composition comprising a potassium channel inhibitor includes a therapeutically effective amount of at least one other therapeutic agent, and a pharmaceutically acceptable excipient.

In one exemplary embodiment, an additional therapeutic agent is a chondrogenesis stimulating agent such as, e.g., interleukin agonists, including IL-4, IL-10, IL-13 agonists, growth factors such as, e.g., TGF-β1, TGF-β2, and TGF-β3; bone morphogenetic protein agonists such as, e.g., BMP-2, BMP-4, BMP-6, BMP-7, BMP-8, BMP-12, BMP-13; insulin like growth factors such as, e.g. IGF-1; growth and differentiation factors (GDFs); wingless type family members (WNTs); Hedgehogs; melanoma inhibitory activity (MIA); a retinoic acid receptor (RAR) antagonist; and fibroblast growth factors such as, e.g., bFGF. For additional chondrogenesis inducing agents, see, e.g., U.S. Pat. No. 6,849,606, incorporated herein by reference. In other embodiments, the second therapeutic agent is a chondrogenic protective agent such as, e.g., IL-1 receptor antagonists, TNF receptor antagonists, COX-2 inhibitors, MAP kinase inhibitors, nitric oxide synthase inhibitors, NFkB inhibitors, viscosupplements (i.e. hyaluronic acid), glucosamine, or chondroitin sulphate. In certain embodiments, the pharmaceutical composition comprising a potassium channel inhibitor also includes a therapeutically effective amount of both a chondrogenesis stimulating agent and a chondrogenic protective agent. Alternatively, a chondrogenic composition comprising a potassium channel inhibitor may be administered with a separate chondrogenesis stimulating agent and/or a chondrogenic protective agent, either simultaneously or sequentially.

The chondrogenic compositions comprising a potassium channel inhibitor may also be used in combination with cells that have chondrogenic potential, including stem cells, progenitor cells, mesenchymal stem cells, mesenchymal progenitor cells, chondroprogenitors, chondrocytes or dedifferentiated chondrocytes.

In certain embodiments, the chondrogenic compositions of the invention may also exhibit chondroprotective activity. A composition can be evaluated for chondroprotective activity by a variety of means known in the art, including direct visualization of cells, histological grading of cartilage, or by molecular diagnostics, such as monitoring the expression level of one or more genes involved in cartilage metabolism. For example, the Mmp13 gene product (human, mouse, and rat GeneIDs: 4322, 17386, 171052, respectively) has been implicated in cartilage degeneration. Neuhold et al., J. Clin. Invest 107(1): 35-44 (2001). Accordingly, in some embodiments, a compound's ability to reduce Mmp13 activity is indicative of chondroprotective activity. In some embodiments, a composition with chondroprotective activity reduces Mmp13 activity in a target cell by at least 10, 15, 20, 30, 50, or 90%; or 2, 4, 6, 8, 10, 20, 50, or 80-fold, relative to a control cell. In more particular embodiments, a composition with chondroprotective activity reduces Mmp13 mRNA or protein levels by at least 10, 15, 20, 30, 50, 90% or 2, 4, 6, 8, 10, 20, 50, 80-fold, relative to control cells.

### Therapeutic Uses

The methods of the present invention include induction of chondrogenesis and cartilage healing or regeneration by administration of any of the chondrogenic compositions comprising a potassium channel inhibitor described herein. In certain embodiments, the invention provides methods of treating or repairing injuries to the articular cartilage and repair or augmentation of hyaline cartilage. Also provided are methods of inducing de novo cartilaginous tissue formation by administration of potassium channel inhibitors. These methods contribute to the repair of congenital, trauma induced, or cartilage defects of other origins, and are also useful in surgery for attachment or repair of cartilage. In some embodiments, methods of the invention comprise the treatment and/or prophylaxis of articular cartilage tears, cartilage deformities, damage to hyaline cartilage, and other cartilage defects in humans and animals.

The methods of the invention further provide for the treatment of chondromalacia, chondrodysplasias, cartilage damage due to bone fracture, osteochondritis, congenital cartilage defects, osteochondritis dessecans, articular cartilage damage, herniated intervertebral disk, anotia, microtia cartilage defect, degenerative joint disease including arthritis (rheumatoid and osteoarthritis), and polychondritis by administration of a composition comprising a potassium channel inhibitor. These compositions may provide an environment to attract cartilage-forming cells, stimulate growth of cartilage-forming cells, induce differentiation of progenitors of cartilage-forming cells, or improve fixation of cartilage to bone or other tissues. Thus, the compositions may be employed in tissue engineering of cartilage. The compositions comprising potassium channel inhibitors may also be administered prophylactically to prevent damage to cartilaginous tissue.

The chondrogenic compositions may be administered in the methods of the invention using methods known to those of skill in the art. In particular, the chondrogenic compositions may be delivered systemically by injection, irrigation, ingestion, inhalation, or topical application. Alternatively, the chondrogenic compositions may be administered locally by injection (including intra-articular), topical application, or as a coating or component of a device or implant. The chondrogenic compositions comprising a potassium channel inhibitor may also be employed to treat cells ex vivo prior to implantation pursuant to the methods of the invention.

Suitable doses of potassium channel inhibitors will be readily determined based on the desired outcome, using routine skill in the medical arts (see, for example Physicians' Desk Reference, 58th Edition, Thompson, P D R, Montvale, N.J. 2004). For example, effective doses identified through in vitro or ex vivo assays can be calibrated for use in vivo, while doses effective in in vivo animal models can be readily interconverted between species by means known in the art (see, for example, Freienreich et al., Cancer Chemother. Rep. 50(4):219-44 (1966)). In some embodiments, suitable doses of potassium channel inhibitors are at least 0.01, 0.05, 0.1, 0.5, 1, 2, 5, 10, 15, 20, 30, 45, 50, 80, 100, 150, 200, 400, 600, 800, 1000, 2000, 4000, or 8000 μM. These doses may be used ex vivo (e.g., for chondrocyte transplantation) or in vivo for direct administration. Doses may also be calculated based on subject's body weight, e.g., at least 0.001, 0.005, 0.01, 0.05, 0.1, 0.5, 1, 5, 10, 20, 40, 80, 100, 200, 400, 800, 1000, 2000, 4000, 8000, 10000, 20000, 40000, or 80000 μg/kg. Dosing regimens may be carried out for a period of time sufficient to achieve the desired therapeutic outcome, e.g., at least 2, 4, 6, 12, 24, 48, 72 hours, or 4, 5, 10, 15, 20, or 30 days.

### Carriers and Implants

Carriers may aid in forming a composition that possesses appropriate handling characteristics for injectable application to the site of cartilage defect or damage. Adding the potassium channel inhibitor composition to a carrier or implant allows it to remain in the diseased or lesioned site for a time sufficient to allow the composition to increase the regenerative chondrogenic activity of the infiltrating mammalian progenitor or other cells, and to form a space in which new tissue can grow and allow for ingrowth of cells. The carrier may also allow the composition to be released from the disease or lesion site over a time interval appropriate for optimally increasing the rate of regenerative chondrogenic activity of the progenitor cells.

In certain embodiments, the chondrogenic compositions described herein may be administered using an appropriate implantable matrix, carrier, or device. For instance, the implant may provide a surface for cartilaginous tissue formation and/or other tissue formation (e.g., in mediation of bone growth or repair). Some embodiments include implantable mechanical physical devices, biodegradable carriers, biodegradable synthetic carriers, prostheses, demineralized allogenic bone and demineralized xenogenic bone. The implantable matrix, carrier, or device may provide slow release of the potassium channel inhibitor and/or the appropriate environment for presentation thereof. Biodegradable materials, such as cellulose films, or surgical meshes, may also serve as matrices. Such materials could be sutured into an injury site, or wrapped around the cartilage. Some matrices include collagen-based materials, including sponges, such as Helistaf (Integra LifeSciences, Plainsboro, N.J.), or collagen in an injectable form.

One family of carriers that may be used in the methods of the invention comprises collagenous materials, and can be in a form suitable for injection, such as a gel. Such gels may be crosslinked or non-crosslinked. Other forms of collagen, such as dispersions or fibrillar collagen, may also be useful in the methods of the present invention. Another family of carriers includes cellulosic materials such as alkylcellulose, including hydroxyalkylcellulose, methylcellulose, ethylcellulose, hydroxyethylcellulose, hydroxypropylcellulose, hydroxypropylmethylcellulose, and carboxymethylcellulose (CMC).

In some embodiments using cellulosic carriers and collagen gels, the carrier may be in the form of a hydrated cellulosic viscous gel. Viscosity may be increased through mechanical means, such as high agitation for a suitable period of time, followed by autoclaving, or chemically. The active agent and cellulosic carrier may be formulated in a solution of a suitable buffer.

Another class of materials for injectable carriers is resorbable hydroxyapatites, as well as minerals, ceramics and phosphates. Resorbable hydroxyapatites, for example, can be formulated at various porosities with varying resorption rates; their handling characteristics vary from hard implantable types, to gel-like consistency, to those that are injectable but harden at body temperature. Suitable hydroxyapatite and ceramic carriers are described, for example in WO96/36562; and U.S. Pat. Nos. 5,543,019; 5,306,305; 5,258,044; 5,496,399; 5,455,231; 5,336,264; 5,178,845; 5,053,212; 5,047,031; 5,129,905; 5,034,059; 4,880,610; 5,290,763; and 5,563,124; all of which are incorporated herein by reference.

In other embodiments, the carrier is an injectable polymer, which may be viscous and which may optionally include a sequestering agent. Suitable polymers and sequestering agents include those described in U.S. Pat. No. 5,171,579, incorporated herein by reference. Other polymers include pluronics, which are liquid (and hence syringeable) at 4° C. and gel at body temperature. The pluronic Poloxamer 407, MW 12,500, is excreted unchanged in the urine after systemic absorption and has supposedly been shown to be non-toxic in animals. In certain embodiments, the polymer may be a polylactide and/or polyethylene glycol, including poly(lactide)/poly(ethylene glycol) gel. Polylactides may be dissolved in polyethylene glycols, such as low molecular weight (2000) PLA dissolved in PEG to produce a syringeable solution that precipitates PLA upon injection into an aqueous environment, resulting in a relatively firm gel.

In some embodiments, the chondrogenic composition may include a sequestering agent such as hyaluronic acid, sodium alginate, poly(ethylene glycol), polyoxyethylene oxide, carboxyvinyl polymer or poly(vinyl alcohol), or a cellulosic material, such as hydroxycellulose or carboxymethylcellulose. The above materials disclosed to be useful as sequestering agents may themselves be useful as carriers for injection. In addition, combinations of the above described materials may be used.

The choice of a carrier material will be based on biocompatibility, biodegradability, mechanical properties, cosmetic appearance and interface properties. The particular application of the chondrogenic compositions will define the appropriate formulation. Potential matrices for the compositions may be biodegradable and chemically defined. Other matrices may be comprised of pure proteins or extracellular matrix components. Additional potential matrices are non-biodegradable and chemically defined.

### Chondrocyte Transplantation

In certain embodiments, chondrocytes or chondrocyte precursor cells may be implanted along with any of the chondrogenic compositions described herein. Some embodiments include culturing the cells with a composition comprising a potassium channel inhibitor ex vivo, prior to implantation at a defect site. In some embodiments, the chondrocytes or precursor cells may be harvested from the subject in need of treatment (e.g., autologous chondrocyte transplantation). The chondrocytes can be implanted along with a matrix, carrier or device. An autologous bone graft may also be implanted with the chondrocytes for certain subjects in need of bone repair.

In other embodiments, genetically engineered cells may be administered along with any of the chondrogenic compositions described herein, and optionally in combination with an appropriate matrix or carrier that can provide a surface for cartilage and/or other connective tissue growth. The cells may be engineered to express proteins, growth factors, extracellular matrix materials or other chondrogenesis stimulating agents. In some embodiments, various collagenous and non-collagenous proteins are expected to be upregulated and secreted from the engineered cells. This phenomenon accelerates tissue regeneration by enhancing extracellular matrix deposition, and can enhance the engraftment and attachment of transplanted cells into the defect site. A carrier or implantable matrix may be used to provide slow release of the chondrogenic composition and/or differentiated cells.

Other embodiments of the invention will be apparent to those skilled in the art from consideration of the specification and practice of the invention disclosed herein. It is intended that any of the potassium channel inhibitors described herein may be used in any of the formulations described to treat any of the conditions described herein. It is further intended that the specification and examples be considered as exemplary only, with a true scope and spirit of the invention being indicated by the following claims.

### EXAMPLES

The following examples illustrate various embodiments of the invention and are not intended to limit the scope of the invention.

### Example 1

### Establishment and Staining of Primary Limb Mesenchymal Cultures

Primary limb mesenchymal (PLM) cultures were established from CD-1 murine embryonic limbs (E11.5) as previously described (Hoffman et al., J. Cell 174:101-13 (2006); Weston et al., J. Cell Biol., 158:39-51 (2002)). Limb mesenchyme was dissociated by dispase treatment and a single cell suspension was obtained by filtration through a 40 mM cell strainer (BD Biosciences). PLM cells were pelleted by centrifugation at 200×g and resuspended to produce a stock cell suspension at a concentration of 2.0×10⁷ cells/ml. Cells were used for transfection (Example 2) or for establishment of cultures for alcian blue staining.

For the latter, 10 μl of PLM cells were spotted into the well of a 24-well plate, and allowed to adhere for 1 h. Culture medium consisting of 60% Ham's F12 nutrient mix/40% Dulbecco's modified Eagle's medium (DMEM) and supplemented with 10% FBS (Qualified, Invitrogen) was added to each well; this time was considered T=0. Cultures were maintained for a period of up to 4 days, and culture media was replaced on alternate days to minimize handling.

For alcian blue staining, culture medium was aspirated and cells were washed once with PBS. Cultures were fixed in 95% ethanol at −20° C. overnight. Fixative was removed by aspiration and cells were sequentially washed once with PBS, followed by 0.2M HCl. Cells were stained overnight with a 1% alcian blue solution prepared in 0.2M HCl.

### Example 2

### Primary Cell Transfections and Expression

Stock plasmid DNAs were standardized to a concentration of 1 mg/ml. For co-transfections, a ratio of 3:1 gene of interest to reporter gene was used. Luciferase reporter genes consist of a firefly reporter gene, pGL3(4×48), and a Renilla (Renilla reniformis) luciferase reporter phRL-SV40 to normalize for transfection efficiency. Transfections were carried out using Effectene reagent (Qiagen). The pGL3(4×48) reporter contains four repeats of a Sox5/6/9 binding site. Reporter plasmids containing Sox5/6/9 binding sites (pGL3[4×48]) were previously described (Weston et al., J. Cell Biol., 158:39-51 (2002)).

To follow the expression of transcripts for Kcnd2 and Sox9, quantitative real-time PCR was performed using the 7500 Fast Sequence Detection System (Applied Biosystems). The primer/probe set used for detection of Sox9 was as described in Weston et al., J. Cell Biol., 158:39-51 (2002). For detection of all other transcripts, TaqMan Gene Expression Assays (Applied Biosystems) were used. Total RNA was isolated from primary cultures, and an aliquot was reverse transcribed to cDNA using a High Capacity cDNA Archive kit (Applied Biosystems). Quantification was performed using ˜10 ng of total RNA and the expression of all genes relative to endogenous rRNA was determined using TaqMan Ribosomal Control Reagents (Applied Biosystems).

### Example 3

### siRNA Knockdown in PLM Cultures

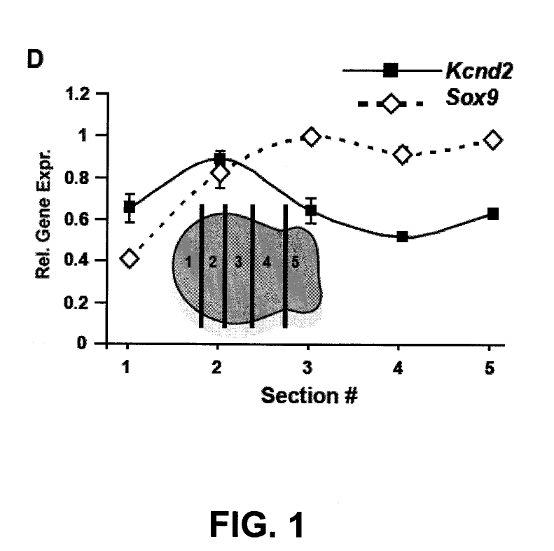

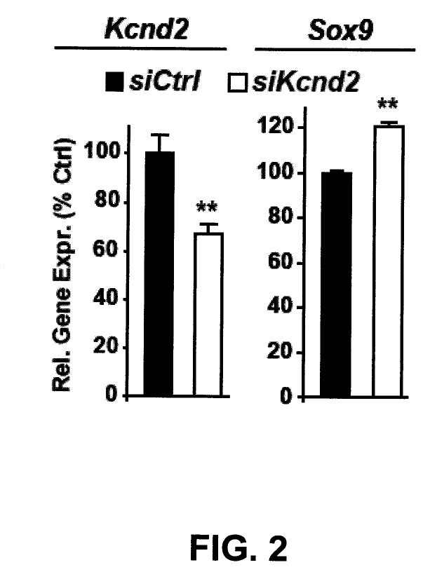

In the developing limb, Kcnd2 and Sox9 are dynamically expressed and an increase in Sox9 expression is preceded by a decrease in Kcnd2 expression (FIG. 1). Kcnd2 knockdown was performed using siRNAs purchased from Dharmacon, and transfected into PLM cells using Lipofectamine™ RNAiMAX (Invitrogen). PLM cells were transfected in suspension with siKcnd2 and 10 μl PLM cultures were established as outlined above. For experiments involving the collection of RNA, siRNA 12-15 transfected PLM cultures were plated per well of a 6-well plate (Nunc), and 2 ml of media were added one hour post-plating. siRNA knockdown of Kcnd2 increases Sox9 expression in PLM cultures (FIG. 2). Achieving a knockdown efficiency of −35% resulted in a 20% increase in Sox9 expression.

### Example 4

### Pro-Chondrogenic Activity of Butamben

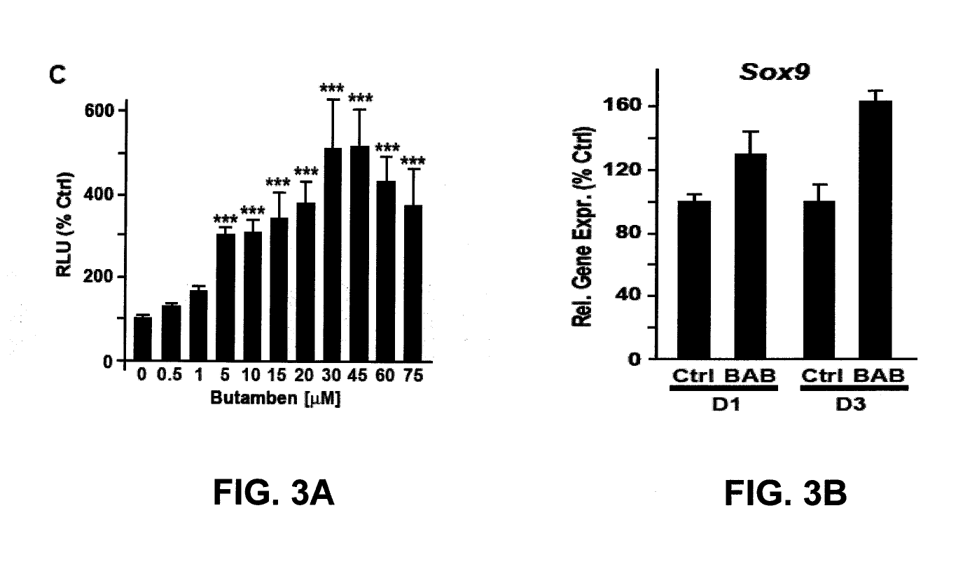

Using procedures based on those described above, the analgesic butamben (butyl 4-aminobenzoate) was found to significantly increase the activity of the chondrogenic responsive reporter gene pGL3(4×48), indicating that it is a chondrogenesis stimulating agent. FIG. 3A shows 4×48 reporter activity (percent activity relative to control) for various doses of butamben. Treatment of PLM cultures with butamben for 1 or 3 days also increased Sox9 expression, as determined by RT-qPCR (FIG. 3B), similar to siKcnd2 knockdown results. According to PLM transfection assays (FIG. 3A) and alcian blue staining experiments, low micromolar concentrations of butamben stimulate chondrogenic activity. This dose range is consistent with previous studies on butamben binding and inhibition of potassium channels.

### Example 5

### Inhibition of Potassium Channels Stimulates Chondrogenesis

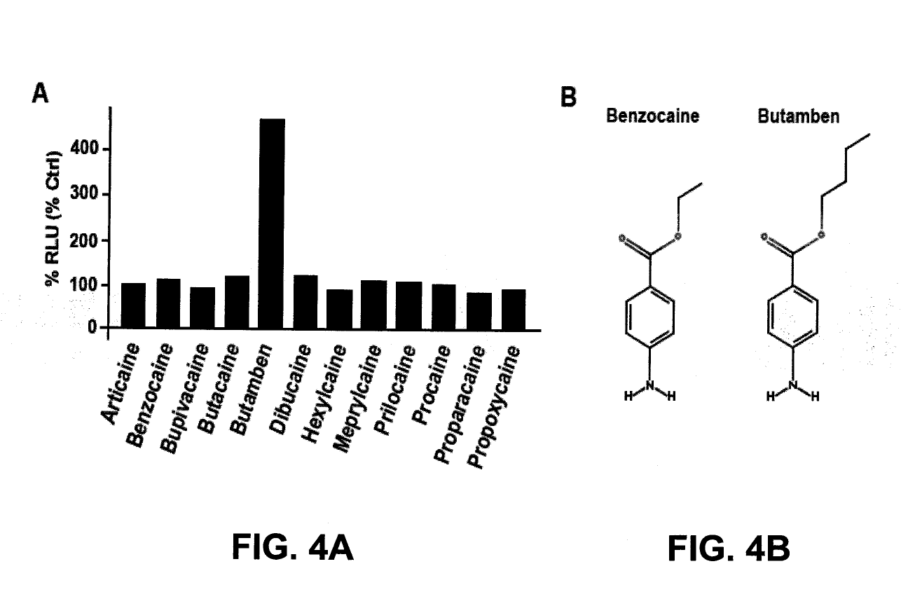

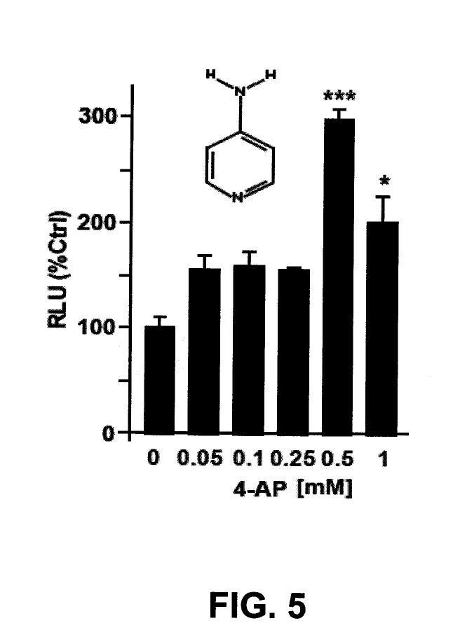

Additional compounds in the benzocaine analgesic family did not significantly increase reporter gene activity, including those with high structural similarity (FIG. 4). While benzocaine analgesics generally function as sodium channel modulators, butamben also exhibits potassium channel inhibitory activity, and blocks the voltage-gated potassium channel Kv1 and 4 classes (Winkelman et al., J. Pharmacol. Exp. Ther., 314:1177-86 (2005); Beekwilder et al., J. Pharmacol. Exp. Ther. 304:531-8 (2003)). To confirm that potassium channel inhibition indeed stimulates chondrogenesis, 4-aminopyridine (4-AP), a broad-spectrum potassium channel blocker, was used in the PLM transfection assay described above. FIG. 5 demonstrates that 4-AP also significantly induces pGL3(4×48) activity.

### Example 6

### Combinations of Butamben and Pro-Chondrogenic Compounds Increase Pro-Chondrogenic Activity

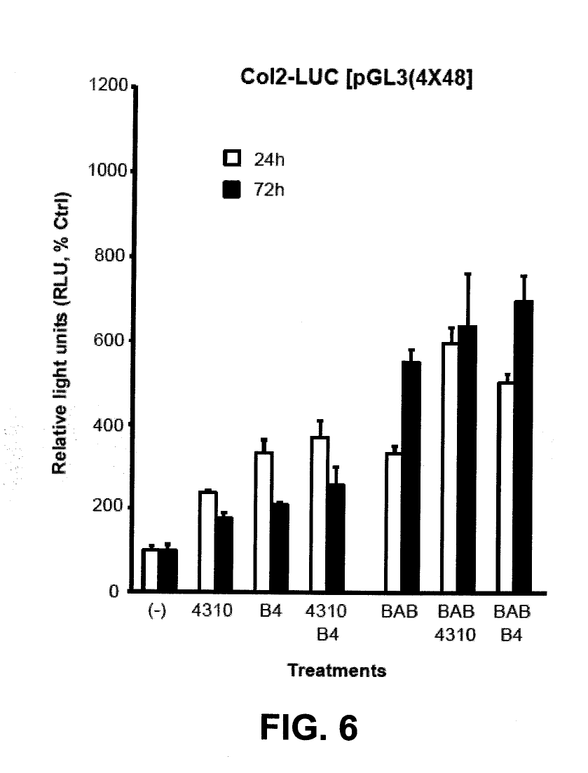

High-density murine limb bud-derived cultures were established as previously described (Hoffman 2006). Media was replaced every two days. Cells were transfected on day 0 with pGL3(4×48) (reporter) and phRL-SV40 (to normalize transfection efficiency) plasmids using Effectene transfection reagent (Qiagen). Twenty-four hours after transfection, cells were treated with the following final concentrations of compounds, either individually or in combination: BMP4 (20 ng/ml), butamben (BAB) (10 μM), and the pan-RAR antagonist 4310 (100 nM). Lysates were collected at 24 h and 72 h after treatment. Luciferase activity was measured using the Dual-Luciferase® kit according to the manufacturer's instructions (Promega). Reporter activity was normalized to untreated controls (−) on the same day. Results from a representative experiment performed in triplicate are shown in FIG. 6.

BAB, 4310, and BMP4 all increased reporter gene activity individually. Reporter gene activity was further increased by combinations of BAB with 4310 or BMP4. This enhanced effect was more pronounced at 24 h. These results demonstrate that BAB, either alone or in combination with BMP4 or 4310, significantly increases pGL3(4×48) reporter activity, indicating its effectiveness as a pro-chondrogenic compound.

### Example 7

### Butamben and pan-RAR Antagonist 4310 Reduce Mmp13 Expression in Limb Mesenchymal Cultures

MMP13 has been identified as a major contributor to cartilage degradation in osteoarthritis, and inhibiting its activity is of therapeutic interest. RAR antagonists promote chondrocyte differentiation and inhibit expression of a hypertrophic phenotype. BMPs stimulate both chondrogenesis and chondrocyte hypertrophy. Accordingly, in this study, 4310 and BMP4 were used as negative and positive controls for chondrocyte hypertropertrophy-inducing activity, respectively.

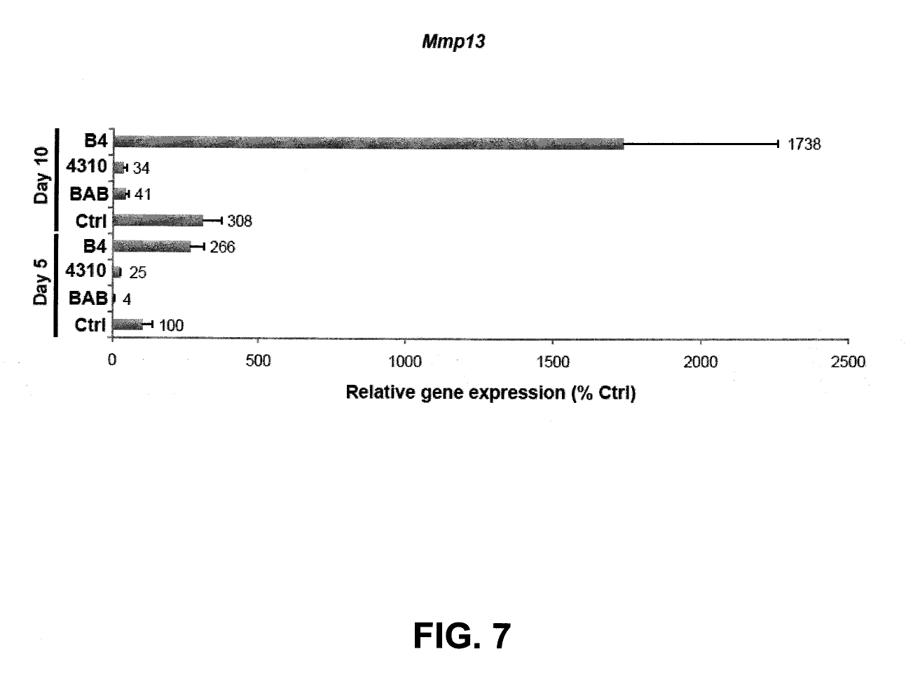

Murine limb bud-derived high-density cultures were established as previously described (Hoffman 2006). Media was replaced every three days. After 24 hours of culture, compounds were added at the following final concentrations: BMP4 (20 ng/ml), BAB (10 μM), and 4310 (100 nM). After 72 hours of culture, ascorbic acid and beta-glycerol phosphate were added to the culture at final concentrations of 126 μM and 1 mM, respectively. RNA was isolated from cultures at five and ten days. Gene expression was assessed using real-time PCR with TaqMan® probe and primer sets (Applied Biosystems, Taqman Gene Expression Assay Mm00439491_m1). All gene expression levels were normalized to the 5-day control (set at 100%). Results are shown in FIG. 7 and are the average of two replicates.

Control cells exhibited an approximately three-fold increase in Mmp13 expression between 5 and 10 days. Consistent with previous reports, BMP4 increased the expression of Mmp13. BAB and 4310 both markedly decreased the expression of Mmp13, indicating that they have chondroprotective activity. Thus, these compounds are attractive alternatives to decrease MMP13 activity, without the side-effects associated with MMP inhibitors.

1. A method for stimulating chondrogenesis or for preventing chondrocyte hypertrophy or maturation or for treating a chondrogenic disease, the method comprising administering to a subject in need thereof a composition comprising a therapeutically effective amount of a potassium channel inhibitor.

2-3. (canceled)

4. The method of claim 1, wherein the chondrogenic disease is selected from the group consisting of chondromalacia, chondrodysplasias, osteochondritis, congenital cartilage disease, osteochondritis dessecans, degenerative or fibrotic joint disease, rheumatoid arthritis, osteoarthritis, and polychondritis.

5. A method for treating or repairing a cartilage defect or for treating, ameliorating or repairing a skeletal defect, a large segmental skeletal gap, or a non-union fracture arising from trauma or surgery, the method comprising administering to a subject in need thereof a composition comprising a therapeutically effective amount of a potassium channel inhibitor.

6. The method of claim 5, wherein the cartilage defect is selected from the group consisting of articular cartilage tears, congenital cartilage defects, and cartilage damage induced by bone fractures.

7. (canceled)

8. The method of claim 5, wherein the composition is provided at the site of the surgery or at the site of the segmental skeletal gap or non-union fracture, and wherein the composition mediates the formation of new bone tissue.

9. (canceled)

10. A method for the ex vivo engineering of chondrocytes comprising:
(a) culturing a population of precursor cells of chondrocyte lineage with a composition comprising a potassium channel inhibitor for a time sufficient to stimulate chondrogenesis; and
(b) implanting the cells from (a) into a desired site in a subject.

11. The method of claim 10, wherein the cells from step (a) are applied to an implantable device selected from the group consisting of a mechanical physical device, biodegradable carrier; biodegradable synthetic carrier, prostheses, demineralized allogenic bone and demineralized xenogenic bone, and implanted into the desired site.

12. The method of claim 1, wherein the potassium channel inhibitor blocks the Kv1, Kv2, Kv3, Kv4, or Kcne classes of potassium channel.

13. The method of claim 5, wherein the potassium channel inhibitor blocks the Kv1, Kv2, Kv3, Kv4, or Kcne classes of potassium channel.

14. (canceled)

15. The method of claim 1, wherein the potassium channel inhibitor is a broad spectrum potassium channel inhibitor.

16. The method of claim 1, wherein the potassium channel inhibitor is selected from the group consisting of butamben, 4-AP, glimepiride, nateglinide, neutralizing antibodies to Kv class potassium channels, and arachidonic acid.

17. The method of claim 5, wherein the potassium channel inhibitor is selected from the group consisting of butamben, 4-AP, glimepiride, nateglinide, neutralizing antibodies to Kv class potassium channels, and arachidonic acid.

18. The method of claim 1, wherein the subject is a mammal.

19. The method of claim 1, wherein the composition is administered systemically, locally, by injection, or as a coating on a device or implant.

20-22. (canceled)

23. The method of claim 1, wherein said composition is used in conjunction with an implantable device selected from the group consisting of a mechanical physical device, biodegradable carrier, biodegradable synthetic carrier, prostheses, demineralized allogenic bone, and demineralized xenogenic bone.

24. The method of claim 1, wherein the composition further comprises another therapeutic agent.

25. The method of claim 24, wherein the therapeutic agent is a second chondrogenesis stimulating agent or is a chondroprotective agent.

26. The method of claim 25, wherein the second chondrogenesis stimulating agent is selected from the group consisting of BMPs, GDFs, FGFs, WNTs, Hedgehog, MIA, and a retinoic acid receptor antagonist.

27. (canceled)

28. The method of claim 25, wherein the chondroprotective agent is selected from the group consisting of IL-1 receptor antagonists, TNF receptor antagonists, COX-2 inhibitors, MAP kinase inhibitors, nitric oxide synthase inhibitors, NFkB inhibitors, hyaluronic acid, glucosamine, and chondroitin sulphate.

29. The method of claim 1, wherein the composition is administered simultaneously or sequentially with a chondrogenesis stimulating agent and/or a chondroprotective agent.

30-43. (canceled)
